SEQUENCE LISTING (6 sequences)

SEQ ID NO: 1 - protein, 9 aa, Artificial
  synthetic peptide
VMAPRTLLL

SEQ ID NO: 2 - protein, 9 aa, Artificial
  synthetic peptide
VMAPRTLVL

SEQ ID NO: 3 - protein, 9 aa, Artificial
  synthetic peptide
VMAPRTLIL

SEQ ID NO: 4 - protein, 9 aa, Artificial
  synthetic peptide
VMAPRTLVL

SEQ ID NO: 5 - protein, 9 aa, Artificial
  synthetic peptide
VTAPRTLLL

SEQ ID NO: 6 - protein, 9 aa, Artificial
  synthetic peptide
VTAPRTVLL

(19) United States
(12) Patent Application Publication
(10) Pub. No.: US 2015/0192570 A1
(43) Pub. Date: Jul. 9, 2015

(54) METHODS FOR DETERMINING WHETHER A CYTOMEGALOVIRUS INFECTION IN A TRANSPLANTED PATIENT IS SUCEPTIBLE TO INDUCE ALLOGRAFT REJECTION

(71) Applicants: INSERM (INSTITUT NATIONAL DE LA SANTE ET DE LA RECHERCHE MEDICALE), Paris (FR); UNIVERSITE DE NANTES, Nantes (FR); CHU NANTES, Nantes (FR); CENTRE NATIONAL DE LA RECHERCHE SCIENTIFIQUE (CNRS), Paris (FR); UNIVERSITE D'ANGERS, Angers (FR)

(72) Inventors: Nadine Gervois, Nantes Cedex 1 (FR); Beatrice Charreau, Nantes (FR); Mathilde Allard, Nantes (FR)

(21) Appl. No.: 14/413,512

(22) PCT Filed: Jul. 9, 2013
(86) PCT No.: PCT/EP2013/064450

§ 371 Date: Jan. 8, 2015

(30) Foreign Application Priority Data
Jul. 10, 2012 (EP) 12305823.2

(51) Int. Cl. G01N 33/50

(52) U.S. Cl. CPC G01N 33/5091; G01N 2800/245

(57) ABSTRACT

The present invention relates to a method for determining whether a cytomegalovirus infection in a transplanted patient is susceptible to induce allograft rejection comprising detecting the presence of at least one HLA-E-restricted CD8 αβ T cell population displaying reactivity against peptides derived from the leader sequences of both HCMV-UL40 protein and allogeneic classical HLA-I molecules in a blood sample of the patient, wherein the presence of said populations indicated that the cytomegalovirus infection in the transplant patient is susceptible to induce allograft rejection.

### FIELD OF THE INVENTION

The present invention relates to methods and kits for determining whether a cytomegalovirus infection in a transplanted patient is susceptible to induce allograft rejection.

### BACKGROUND OF THE INVENTION

Transplantation is considered as the treatment of choice for many patients suffering with organ failure, for improving survival and quality-of-life even. However rejection of allograft has always been the major obstacle to transplantation success.

Cytomegalovirus (CMV) is a common opportunistic pathogen that persists for life in the human host after primary infection. While CMV infection of immunocompetent individuals generally results in subclinical diseases, it may cause serious life threatening complications in immunocompromised ones. Consequently, transplant patients with immunosuppressive regimens are particularly prone to CMV disease and it is estimated that up to 75% of all patients undergoing solid organ transplantation experience new infection or reactivation of latent CMV infection(1, 2).

CMV infection has been implicated in the development of both acute and chronic allograft rejection and has been associated with decreased allograft and patient survival(3, 4). Although association between CMV infection and allograft rejection is well admitted, the precise mechanisms involved remain uncertain.

CMV could account for graft rejection by triggering the activation of endothelial cells, which are preferential targets of CMV infection(5-7). This might directly increase the expression of MHC, costimulatory and adhesion molecules on the allograft endothelium through the induction of mediators release such as type I IFN. Then, activated graft's EC may attract and activate recipient's cytotoxic T cells, which can trigger rejection(8). CMV infection could also be implicated in the development of allograft rejection because of cross-reactivity of CMV-specific T cells toward allogeneic HLA molecules as we and others have previously documented(9-12). An alternate mechanism has been suggested by studies reporting the existence in CMV seropositive individuals of CD8 T cells that recognize, in a HLA-E restricted-fashion, peptides derived from the leader sequences of both HCMV-UL40 and allogeneic classical HLA-I molecules(13-15). Consequently, while this HLA-E-restricted T cells potentially mediate protection against CMV infection, they may also promote graft rejection through recognition of peptides derived from allogeneic HLA-I molecules presented by HLA-E on graft cells.

One of the most striking features of the non-classical HLA-I molecule HLA-E is its highly conserved nature. Only two allelic forms exist in the Caucasian population, HLA-E*0101 (HLA-E^{107R}) and HLA-E*0103 (HLA-E¹⁰⁷⁰) that differ at one amino acid position(16). As a consequence, HLA-E-bound peptides are highly restricted, comprising mostly signal peptides derived from others HLA-I proteins(17). Class lb molecules are often considered to have a prominent role in innate immunity. Among this line, surface expression of HLA-E bound to autologous HLA class I derived peptides, indicating the integrity of the MHC I antigen processing machinery and acting as a ligand for CD94-NKG2 receptors, modulate the activation of NK and T cells(18, 19). However, in times of cellular stress or infections, HLA-E is associated with a much more diverse repertoire of peptides, which can be sensed directly by αβ TCR(20, 21). Indeed, several studies in human and mice have highlighted a dual role for unclassical HLA-Ib molecules, in that, like classical HLA-Ia molecules (ie HLA-A/B/C), they can mediate adaptative immune responses to bacteria(22, 23), viruses(13, 24, 25), tumors(26) and self-antigens(27, 28).

Although HLA-E is virtually expressed in all tissues, its surface expression profile is more restricted than that of classical HLA-I molecules. It was previously reported that, HLA-E surface expression in normal nonlymphoid organs is mainly restricted to endothelial cells(29). Upon solid organ transplantation, because graft endothelial cells display MHC-peptide complexes at their surface and come in regular contact with recipient circulating T cells, the endothelium of allografts plays a central role in eliciting immune-mediated rejection(8, 30). However, while HLA-E has been shown to behave as a strong transplantation antigen in rodent models(31), whether HLA-E expressed on human graft's tissues could trigger an allogeneic cellular response remains to be documented.

### SUMMARY OF THE INVENTION

The present invention relates to a method for determining whether a cytomegalovirus infection in a transplanted patient is susceptible to induce allograft rejection comprising detecting the presence of at least one HLA-E-restricted CD8 αβ T cell population displaying reactivity against peptides derived from the leader sequences of both HCMV-UL40 protein and allogeneic classical HLA-I molecules in a blood sample of the patient, wherein the presence of said populations indicated that the cytomegalovirus infection in the transplant patient is susceptible to induce allograft rejection.

### DETAILED DESCRIPTION OF THE INVENTION

Although association between CMV infection and allograft rejection is well admitted, the precise mechanisms involved remain uncertain. Characterization of alloreactive T cells in CMV seropositive kidney transplant patients allowed us to identify a monoclonal HLA-E-restricted CD8 αβ T cell population displaying reactivity against peptides derived from the leader sequences of both HCMV-UL40 and allogeneic classical HLA-I molecules. As HLA-E expression in nonlymphoid organs is mainly restricted to endothelial cells, the inventors investigated the reactivity of this HLA-E-restricted T cell population towards allogeneic endothelial cells. They clearly demonstrated that CMV-committed HLA-E-restricted T cells efficiently recognized and killed allogeneic endothelial cells in vitro. Therefore, while HLA-E-restricted T cells have potential to contribute to the control of CMV infection, they may also directly mediate graft rejection in vivo through recognition of peptides derived from allogeneic HLA-I molecules on graft cells. Moreover, the data indicate that this alloreactivity is tightly regulated by NK receptors, especially by inhibitory KIR2DL2 that strongly prevents TCR-induced activation through recognition of HLA-C molecules. Hence, a better evaluation of the role of CMV-committed HLA-E-restricted T cells in transplantation and of the impact of HLA-genotype, especially HLA-C, on their alloreactivity may represent a risk factor following organ transplantation.

Accordingly, the present invention relates to a method for determining whether a cytomegalovirus infection in a transplanted patient is susceptible to induce allograft rejection comprising detecting the presence of at least one HLA-E-restricted CD8 αβ T cell population displaying reactivity against peptides derived from the leader sequences of both HCMV-UL40 protein and allogeneic classical HLA-I molecules in a blood sample of the patient, wherein the presence of said populations indicated that the cytomegalovirus infection in the transplant patient is susceptible to induce allograft rejection.

The patient may be transplanted with any type of solid grafts. Grafts of interest include, but are not limited to: transplanted heart, kidney, lung, liver, pancreas, pancreatic islets, brain tissue, stomach, large intestine, small intestine, cornea, skin, trachea, bone, bone marrow, muscle, or bladder.

Cytomegalovirus (CMV) infection may be diagnosed with any standard methods well known in the art. Typically serology provides indirect evidence of recent CMV infection based upon changes in antibody titers at different time points during a clinical illness. Many different antibody detection techniques are available, including complement-fixation techniques, enzyme-linked immunosorbent assays (ELISA), latex agglutination, radioimmunoassays, and indirect hemagglutination assays (Chou S. Newer methods for diagnosis of cytomegalovirus infection. Rev Infect Dis 1990; 12 Suppl 7:S727.). Liquid-phase luciferase immunoprecipitation systems have also been developed to provide qualitative assessments of anti-CMV antibodies (Burbelo P D, Issa A T, Ching K H, et al. Highly quantitative serological detection of anti-cytomegalovirus (CMV) antibodies. Virol J 2009; 6:45). Accordingly, the patient is typically a cytomegalovirus-seropositive transplant patient.

The term “blood sample” means a whole blood sample obtained from the patient. Typically, the peripheral blood mononuclear cells (PBMCs) are isolated by Ficoll-density gradient centrifugation.

Standard methods well known in the art may be used for detecting the presence of at least one the HLA-E-restricted CD8 αβ T cell population of the invention in the blood sample. For example, standard methods for detecting the expression of the specific surface markers of the population may be performed. The inventors indeed demonstrated that the population is typically characterized by the classical surface markers of T CD8 cells (e.g. CD3 and CD8) and by the expression of the Killer-Cell Immunoglobulin-like Receptor (KIR) KIR2DL2. The population may also be characterized by CD8αβ⁺CD62L⁻CCRTCD27⁻ CD28^{+/-}CD45RA^{lo}CD45RO^{hi}CD57⁻ surface phenotype and the surface expression of ILT-2, NKG2-D and CD94. Accordingly, the detection of at least one HLA-E-restricted CD8 αβ T cell population of the invention may be determined by using a set of binding partners directed against certain surface marker of the invention (e.g. CD8 and KIR2DL2).

As used herein, the term “binding partner directed against the surface marker” refers to any molecule (natural or not) that is able to bind the Surface marker with high affinity. Said binding partners include but are not limited to antibodies, aptamer, and peptides. The binding partners may be antibodies that may be polyclonal or monoclonal, preferably monoclonal, specifically directed against said Surface marker. In another embodiment, the binding partners may be a set of aptamers.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies of the invention or a fragment thereof can be prepared and isolated using any technique that provides for the production of antibody molecules by continuous cell lines in culture. Techniques for production and isolation include but are not limited to the hybridoma technique originally; the human B-cell hybridoma technique; and the EBV-hybridoma technique. In a particular embodiment, antibodies as described in the EXAMPLE may be used.

In another embodiment, the binding partners may be aptamers. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence.

The binding partners of the invention such as antibodies or aptamers may be labelled with a detectable molecule or substance, such as preferentially a fluorescent molecule, or a radioactive molecule or any others labels known in the art. Labels are known in the art that generally provide (either directly or indirectly) a signal.

As used herein, the term “labelled”, with regard to the antibody or aptamer, is intended to encompass direct labelling of the antibody or aptamer by coupling (i.e., physically linking) a detectable substance, such as a fluorophore (e.g. Fluorescein Isothiocyanate (FITC) or Phycoerythrin (PE) or Allophycocyanin (APC) or Allophycocyanin-Cyanin7 (APC-H7) or Brilliant Violet 421) or a radioactive agent to the antibody or aptamer, as well as indirect labelling of the probe or antibody by reactivity with a detectable substance. Preferably, the antibodies against the Surface marker are already conjugated to a fluorophore (e.g. FITC-conjugated and/or PE-conjugated).

The aforementioned assays may involve the binding of the binding partners (ie. antibodies or aptamers) to a solid support. The solid surface could be a microtitration plate coated with the set of binding partners. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In a preferred embodiment, fluorescent beads are those contained in TruCount(™) tubes, available from Becton Dickinson Biosciences, (San Jose, Calif.). According to the invention, methods of flow cytometry are preferred methods for measuring the level of Surface markers at the platelet surface. Said methods are well known in the art. For example, fluorescence activated cell sorting (FACS) may be therefore used as described in the EXAMPLE.

In one embodiment, the reactivity of at least one HLA-E-restricted CD8 αβ T cell population against peptides derived from the leader sequences of HCMV-UL40 protein and allogeneic classical HLA-I molecules may be further determined according to any well known method in the art. For example once isolated, the population may be cultured as described in the EXAMPLE and various assays may be used to determine the alloreactivity against the leader sequences of HCMV-UL40 protein and allogenic classical HLA-I molecules. Typically, a B-EBV cell line (i.e a B cell immortalized with Epstein Barr virus) transected with a nucleic acid molecule encoding for a HLA-E polypeptide (preferably together with a leader sequence peptide from a HLA-B polypeptide) may be used. The cell line is then pulsed with at least one UL40 peptide as above described and incubated with the isolated CD8 αβ T cell population of the invention. The production of at least one cytokine or interleukin (e.g. TNF-alpha, IFN-gamma, . . . ) may be finally assessed, and it is concluded that the isolated HLA-E-restricted CD8 αβ T cell population of the invention is reactive against peptides derived from the leader sequences of HCMV-UL40 protein (different human CMV strains) when the production of the cytokine or interleukin is detected. Such assays are typically described in the EXAMPLE. Alternatively HLA-E tetramer refolded with an alloreactive HCMV-UL40 peptide may be used as described in the EXAMPLE for determining whether the populations of the invention is able to recognize the leader sequence of both HCMV-UL40 protein. The UL40 peptide may be selected from the group consisting of VMAPRTLLL (SEQ ID NO:1), VMAPRTLVL (SEQ ID NO:2) and VMAPRTLIL (SEQ ID NO:3).

The method of the invention may further comprise determining the HLA class I typing of transplant donor. Data on the HLA typing of transplant donor may inform about i) the presence of one or several HLA allele(s) whom signal sequence is (are) the same as above mentioned CMV sequences, susceptible to be recognize by CMV-committed HLA-E-restricted CD8 αβ T cells and ii) the presence of one or several HLA-C allele(s) corresponding to KIR2DL2 ligand. Indeed the presence of at least one monoclonal HLA-E-restricted CD8 αβ T cell population of the invention with the presence of one or several HLA allele(s) whom signal sequence is (are) the same as above mentioned CMV sequences associated with the absence of a HLA-C ligand for KIR2DL2 in the graft organ indicate that CMV infection is highly susceptible to induce graft rejection (see Table A-C). Methods for determining the HLA class I haplotype (of donors and recipients) and are well known in the art and may be performed on blood samples and involve use of HLA-Class I antibodies or multiplex PCR reactions.

TABLE A
graft risk rejection considerations when a HLA-E-restricted
CD8 αβ T cell population displaying reactivity against
VMAPRTLLL (SEQ ID NO: 1) is detected in combination
with the HLA typing of transplant donor:

| HLA typing of transplant donor | Graft rejection risk |
|---|---|
| HLA-A*01, -A*03, -A*11, -A*29, | High risk of graft rejection |
| -A*30, -A*31, -A*32, -A*33, -A*36 | |
| -A*74, -Cw*2 and -Cw*15 with the | |
| absence of a HLA-C ligand for | |
| KIR2DL2 (HLA-Cw3 and related, | |
| ‘group1’ alleles) | |

TABLE B
graft risk rejection considerations when a HLA-E-restricted
CD8 αβ T cell population displaying reactivity against
VMAPRTLVL (SEQ ID NO: 2) is detected in combination
with the HLA typing of transplant donor:

| HLA typing of transplant donor | Graft rejection risk |
|---|---|
| HLA-A*02, -A*23, -A*24, -A*25, | High risk of graft rejection |
| -A*26, -A*3402, -A*43, -A*66 and | |
| -A*69 with the absence of a HLA-C | |
| ligand for KIR2DL2 (HLA-Cw3 and | |
| related, ‘group1’ alleles) | |

TABLE C
graft risk rejection considerations when a HLA-E-restricted
CD8 αβ T cell population displaying reactivity against
VMAPRTLIL (SEQ ID NO: 3) is detected in combination
with the HLA typing of transplant donor:

| HLA typing of transplant donor | Graft rejection risk |
|---|---|
| HLA-Cw*01, -Cw*03, -Cw*0401, | High risk of graft rejection |
| -Cw*05, -Cw*06, -Cw*0801-03, | |
| -Cw*12, -Cw*14, -Cw*16 and | |
| -Cw*1702 with the absence of a HLA-C | |
| ligand for KIR2DL2 (HLA-Cw3 and | |
| related, ‘group1’ alleles) | |

The present invention also relates to an agent depleting at least one HLA-E-restricted CD8 αβ T cell populations as above described for use in the prophylactic treatment of a patient considered at risk for graft rejection by the method of the invention.

Typically, said agent may be an anti-KIR2DL2 monoclonal antibody or a soluble KIR2DL2 ligand.

The agent of the invention may be combined with pharmaceutically acceptable excipients, and optionally sustained-release matrices, such as biodegradable polymers, to form therapeutic compositions.

In the pharmaceutical compositions of the present invention, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The Agent of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1. Detection and characterization of HLA-E-restricted CD8 T cells in PBL of a kidney transplant patient undergoing CMV infection

A/ PBL reactivity against COS-7 cells transfected, or not, with HLA-I encoding cDNA was assessed by a TNF release assay. Means and standard deviations of sixplicates are shown. B/ TNF production in response to HLA-E transfected .221-E cells in the presence of blocking antibodies. MART.22 was stimulated with target cells in the presence or not of blocking antibodies directed against total HLA-I, HLA-A/B/C, HLA-E, CD3 and CD8 molecules at the indicated concentrations. After 6 h, T cells were fixed, permeabilized and stained for intracellular TNF-α. Results are expressed as percentage of TNF-producing T cells. C/ Time course of TCRαβ, CD3 and CD8 surface expression on HLA-E-restricted CD8 T cells stimulated with .221-E cells. A representative FACS analysis of TCRαβ at early time course is shown (left panel). Results are expressed as percentages of RFI (as defined in Material and Methods).

FIG. 2. Characterization of CMV/HLA-I-derived peptides recognized by HLA-E-restricted CD8 T cells

A/ TNF production in response to stimulation with .221 cells pulsed with synthetic peptides. .221 cells were incubated for 1 h with range concentrations of the indicated peptides before addition of MART.22 T cells. After 6 h, T cells were fixed, permeabilized and stained for intracellular TNF-α. Results are expressed as percentage of TNF-producing T cells. B/ Peptide-MHC tetramer staining of HLA-E-restricted CD8 T cells. MART.22 T cells were incubated for 1 h with biotyniled HLA-E monomers refolded with the indicated peptides and tetramerized with PE-coupled streptavidin. Peptide-HLA-E tetramers staining was assessed by flow cytometry and MFI are indicated.

FIG. 3. Functional characterization of HLA-E-restricted CD8 T cells

A/ Induction of strong and rapid Ca²⁺ responses within activated HLA-E-restricted CD8 T cells. B-EBV 721.221 cells transfected (.221-E) or not (.221) with HLA-E and the leader sequence of HLA-B*08, were incubated with MART.22 T cells loaded with Fura-2 (1:1 ratio). T cell intracellular Ca²⁺ levels were monitored by videomicroscopy for the indicated acquisition time. Graphs represent the kinetics of intracellular Ca2⁺ levels (340/380 nm ratio). Values correspond to the mean of emission measured among all T cells present in the field (approximatively 20 cells per experiment). Results are representative of two independent experiments. B/ Degranulation of HLA-E-restricted CD8 T cells upon stimulation. .221-E cells (thick line) or .221 cells (thin line) were incubated for 4 h with MART.22 T cells in the presence of anti-CD107a antibody. Results are expressed as pourcentages of surface CD107a positives T cells upon stimulation with .221-E cells. C/ Cytotoxic activity of HLA-E restricted CD8 T cells. 10^{3 51}Cr-labeled .221-E cells (squares) or .221 cells (circles) were co-cultured for 4 h with MART.22 T cells at various E/T ratios. Cytotoxic activity was assessed through measure of Chromium release in the supernatants. Percentages of specific lysis are indicated. Means and standard deviations of triplicate wells are shown for one out of three comparable experiments. D/ Cytokine production analysis of HLA-E restricted CD8 T cells. MART.22 T cells were fixed, permeabilized and stained for intracellular cytokines following 6 h of incubation with .221-E cells (thick line) or .221 cells (thin line). Data are expressed as mean % of intracellular cytokine secreting cells upon stimulation with .221-E cells.

FIG. 4. Expression of NK receptors by HLA-E-restricted CD8 T cells and functional characterization

A/ Surface expression of NK receptors by HLA-E-restricted CD8 T cells MFI of stained T cells (thick line) are indicated. B/ Modulation of HLA-E restricted CD8 T cells reactivity through NKR engagement. ⁵¹Cr-labeled P815 cells were preincubated with the indicated concentration of anti-CD3 antibody in the presence or not of the indicated anti-NKR antibody for 1 h. Then, MART.22 T cells were added for 4 h. Redirected cytotoxic activity was assessed through measure of Chromium release in the supernatants. Percentages of specific lysis are indicated. Means and standard deviations of triplicate wells are shown for one representative experiments out of three performed.

FIG. 5. Reactivity of HLA-E-restricted CD8 T cells against allogeneic endothelial cells

A/ Surface expression of HLA-E (thick lines) and total HLA-I (dotted lines) molecules by two representative endothelial cultures (HAEC). MFI are indicated. B/ Cytokine production by HLA-E-restricted CD8 T cells upon stimulation with endothelials cultures. MART.22 T cells were fixed, permeabilized and stained for intracellular TNF-α following 6 h of incubation with HAECs (thick line) or not (thin line). Data are expressed as percentage of intracellular cytokine secreting T cells upon stimulation with HAECs. C/ Degranulation of HLA-E-restricted CD8 T cells upon stimulation with endothelial cultures. MART.22 T cells were incubated for 4 h with HAECs (thick line) or not (thin line) in the presence of anti-CD107a antibody. Results are expressed as percentages of surface CD107a positive T cells upon stimulation with endothelial cells.

FIG. 6. Regulation of HLA-E-restricted CD8 T cells reactivity against allogeneic endothelial cells by NK receptor

A/ Reactivity of HLA-E-restricted T cells against unrecognized endothelial cultures pulsed with synthetic peptides. HAECs were incubated for 1 h with range concentrations of the indicated peptides before MART.22 T cells were added. After 6 h, T cells were fixed, permeabilized and stained for intracellular TNF-α. Results are expressed as percentage of TNF-producing T cells. B/ Impact of KIR2DL2-ligands expression by HAECs on HLA-E-restricted T cells alloreactivity. Percentages of TNF-producing MART.22 T cells are shown for HAECs with none, one or two protective HLA-C alleles. C/ Reactivity of HLA-E-restricted T cells against unrecognized endothelial cultures in the presence of blocking antibodies. HAECs were incubated with MART.22 T cells in the presence or not of indicated concentrations of blocking antobodies. After 6 h, T cells were fixed, permeabilized and stained for intracellular TNF-α. Results are expressed as percentage of TNF-producing T cells.

FIG. 7. Impact of IFN-γ treatment on allogeneic endothelial cells recognition by HLA-E-restricted CD8 T cells

A/ impact of IFN-γ treatment on surface expression of HLA-E (thick lines) and total HLA-I (dotted lines) molecules by endothelial cultures. MFI are indicated. B/ HLA-E-restricted CD8 T cells cytotoxicity toward endothelial cultures treated or not with IFN-γ. 103 ⁵¹ Cr-labeled HAECs pretreated (closed circles) or not (open circle) with IFN-γ were co-cultured for 4 h with T cells at various E:T ratio. Cytotoxic activity was assessed trought measure of Chromium release in the supernatants. Percentages of specific lysis are indicated. Means and standard deviations of triplicate wells are shown for one representative experiments out of three performed. C/ HLA-E-restricted CD8 T cells cytokine production upon stimulation with endothelials cultures treated or not with IFN-γ. MART.22 T cells were fixed, permeabilized and stained for intracellular cytokines following 6 h of incubation with HAECs pretreated (black bars) or not (white bars) with INF-γ. Data are expressed as percentages of intracellular cytokine secreting T cells upon stimulation. D/ HLA-E-restricted CD8 T cells cytokine production upon stimulation with endothelials cultures treated or not by IFN-γ, in the presence of blocking antibodies. MART.22 T cells were fixed, permeabilized and stained for intracellular cytokines following incubation for 6 h with HAECs pretreated or not with IFN-γ in the presence or not of various amount of blocking antibodies directed against KIR2DL2, ILT-2 and HLA-Ia molecules. Data are expressed as percentages of intracellular TNF secreting T cells upon stimulation.

### EXAMPLE

Material & Methods

HLA-E-Restricted CD8 T Cells Isolation and Culture

Blood sample was collected from a CMV-seropositive kidney-transplant patient (HLA-A*0201, -B*4402, -B*5101, -Cw*0501 and -Cw*1402) (referred as KR2 in a previous study)(32) with formal consent. PBMC were isolated by a Ficoll density gradient (PAA, Les Mureaux, France) and cultured with RPMI 1640 (Sigma-Aldrich, Saint-Quentin Fallavier, France) containing 8% human serum (local production) and 150 U/mL rIL-2 (Eurocetus, Rueil-Malmaison, France). HLA-E-reactive population was enriched using a TNF-α Secretion Assay Cell Enrichment and Detection Kit (Miltenyi, Paris, France) after stimulation with HLA-E-transfected COS-7 cells. Sorted cells were cloned by limiting dilution and expanded by stimulation with phytohemagglutinin (PHA)-L (Sigma-Aldrich) in the presence of irradiated feeder cells (allogeneic lymphocytes and Epstein Barr Virus-transformed B lymphocytes)(33).

HAEC Isolation, Culture and IFN-γactivation

Human arterial endothelial cells (HAEC) were isolated from unused artery pieces collected at the time of kidney transplantation, harvested according to good medical practice and stored in the DIVAT Biocollection (French Health Minister Project number 02G55)(34). Briefly, fragment of arteries were incubated with collagenase A (Roche, Basel, Switzerland) for 30 min at 37° C. and EC were selected using CD31-Dynabeads (Dynal, Villebon sur Yvette, France). HAEC were grown in Endothelial Cell Basal Medium (ECBM) supplemented with 10% fetal calf serum (FCS, PAA, France), 0.004 m-L/mL ECGS/Heparin, 0.1 ng/mL hEGF, 1 ng/mL hbFGF, 1 μg/mL hydrocortisone, 50 μg/mL gentamicin and 50 ng/mL amphotericin B (C-22010, PromoCell, Heidelberg, Germany). For activation, confluent HAEC monolayers were starved overnight in ECBM supplemented with 2% FCS without growth factors and incubated with recombinant human IFN-γ (50 U/mL, Imukin, Boehringer Ingelheim, Germany) for 48 h. HLA class I genotyping was performed by the Etablissement Français du Sang (Nantes, France).

B-EBV 721.221 and COS-7 Cells Culture

The HLA-E-transfected (721.221-E) and untransfected (721.221) B-EBV cell lines were kindly provided by V. Braud (UMR CNRS 6097/Université Nice-Sophia Antipolis, Valbonne, France). COS-7 cells were obtained from T. Boon (Ludwig institute for Cancer Research, Brussels, Belgium). These cells were maintained in RPMI 1640 10% FSC.

Antibodies

The following antibodies were used in a conjugated form (phenotyping) or not (blocking or redirected lysis experiments) with fluorescein isothiocyanate (FITC), phycoerythrin (PE) or allophycocyanin (APC): TCRαβ-PE, CD8α-PE, IFN-γ-PE (Miltenyi), CD3-PE, CD27-PE, CD28-PE, CD45-RA-PE, CD45-RO-PE, CD56-PE, CD57-FITC, CD62-L-PE, CCR7-PE, CD107a-PE, Perforine-FITC, Granzyme-A-FITC, TNF-α-PE, GM-CSF-PE, TGF-β-PE, IL-2-PE, IL-4-PE, IL-5-PE, IL-β-PE, IL-21-PE, HLA-A/B/C (clone G46-2.6) (Becton Dickinson, Le Pont de Claix, France), CD8β-PE, CD94-PE (clone HP-3B1), NKG2A-PE (clone Z199), KIR2DS1/2DL1-APC (clone EB6), KIR2DS2/2DL2/2DL3-APC (clone GL183), KIR2DS4 (clone FES172), KIR3DS1/3DL1-PE (clone ZIN273), ILT-2-PE (clone HPF1) (Beckman Coulters,

Villepinte, France), NKG2C-PE (clone 134522), NKG2D-PE (clone 149810), IL-17F-PE (R&D, Lille, France), IL-22-PE, HLA-E (clone 3D12) (BioLegend, San Diego, Calif.) and HLA-I (clone W6.32, American Type Culture Collection).

Peptides and Recombinant Peptide/HLA-E Monomers

Peptides VMAPRTLLL and VMAPRTLVL (HLA-A*01- and HLA-A*02-derived signal peptides respectively) with purity >85% were purchased from Eurogentec (Angers, France). HLA-E*0101/peptide monomers were generated by the recombinant protein facility of SFR26 (Nantes, France).

Phenotypic Characterization by Flow Cytometry

For membrane staining, 2×10⁵ cells were incubated at 4° C. with 10 μg/ml of Ab (specific or isotype control) or tetramers for 30 min or 1 h respectively. When non-conjugated mAb were used, a second incubation with PE-conjugated goat F(ab′)2 fragment anti-Mouse IgG (Beckman Coulters) was performed. 5×10⁴ cells were acquired in the viable cells gate on a FACScalibur flow cytometer using CellQuest software (Becton Dickinson).

Transient Transfection of COS-7 Cells and TNF Assay

Briefly, 20×10³ COS-7 cells were transfected with 100 ng of HLA-E*0101 or HLA-E*0103 encoding plasmid by the DEAE-dextran-chloroquine method. 48 h after transfection, 5×10³ T cells were added to transfected COS-7 cells. Culture supernatants were harvested 6 h later and tested for TNF content through assessment of the sensitive WEHI164 clone13 viability in a MTT colorimetric assay.

Intracellular Staining

For cytokine/perforine/granzyme intracellular staining, 1×10⁵ T cells were stimulated in the presence of Brefeldin A (Sigma-Aldrich, 10 μg/ml) with 2×10⁵ target cells (B-EBV cells or HAEC) for 6 h at 37° C., in the presence or not of blocking Abs. For peptide loading, target cells were incubated with peptides for 1 h at 37° C. before incubation with T cells. Cells were then fixed with 4% paraformaldehyde (Sigma-Aldrich), labeled with specific mAbs and analyzed by flow cytometry.

CD107a Degranulation

1×10⁵ T cells were stimulated with 2×10⁵ target cells in the presence of anti-CD107a mAb. After 4 h at 37° C., cells were analyzed by flow cytometry.

TCR-αβ/CD3/CD8 Downregulation

1×10⁵ T cells were stimulated with 2×10⁵ target cells at 37° C. After the indicated time, TCR-αβ/CD3/CD8 fluorescence intensity was measured in unstimulated and activated lymphocytes. Relative fluorescence intensity (RFI) was calculated as sample mean fluorescence divided by isotype control mean fluorescence. Data were expressed as percentages of RFI that were calculated according to the following formula: (RFI of activated lymphocytes/RFI of unstimulated lymphocytes)×100.

Single-Cell Ca²⁺ Video Imaging

Fura-2/AM loaded T cells (1 μM, Invitrogen, Cergy-Pontoise, France) for 1 h at room temperature in HBSS (Invitrogen) were resuspended in HBSS 1% FCS and seeded on Lab-Tek glass chamber slides (Nunc, Naperville, Ill.) coated with poly-L-lysin (Sigma-Aldrich). Target cells were left to adhere on glass slides before addition of T cells. Measurements of intracellular Ca²⁺ responses were performed at 37° C. with a DMI 6000 B microscope (Leica Microsystems, Nanterre, France). Cells were illuminated every 15 s with a 300 W xenon lamp by using 340/10 nm and 380/10 nm excitation filters. Emission at 510 nm was used for analysis of Ca²⁺ responses and captured with a Cool Snap HQ2 camera (Roper, Tucson, Ariz.) and analyzed with Metafluor 7.1 imaging software (Universal Imaging, Downington, Pa.).

⁵¹Cr Release Assay

Target cells were labeled with 100 μCi Na⁵¹CrO₄ (Oris Industrie, Gif-sur-Yvette, France) for 1 h at 37° C., and incubated 4 h at 37° C., with effectors T cells at various E/T ratios. Then, 25 μl of supernatants were mixed with 100 μl of scintillation liquid (Optiphase Supermix, Wallak, United Kingdom) for measurement of radioactive content on a beta plate counter (EG&G Wallac, Evry, France). Percentage of target cell lysis was calculated according to the following formula: [(experimental release−spontaneous release)/(maximum release−spontaneous release)]×100. Maximum and spontaneous releases were determined by, respectively, adding 0.1% Triton X-100 or medium to ⁵¹Cr-labeled target cells in the absence of T cells.

Redirected Cytolytic Activity

1×10^{3 51}Cr-labeled murine mastocytoma FcγR P815 cells were incubated with T cells at various E/T ratio, in the presence of different concentrations of anti-CD3 Ab (clone OKT3). CD3 redirected lysis of P815 cells was modulated by the presence of indicated anti-NKR Abs (10 μg/ml). After 4 h, measurement of radioactive content and determination of percentage of specific lysis were performed.

CMV-Committed HLA-E-restricted CD8 αβ T Cells Staining Protocol
Prepare PBMC at a concentration of 2.5×10⁶ cells per ml in PBS buffer and add 100 μl to each well of a 98 microwells plate.
Prepare CD94 Blocking cocktail containing 5 μg/ml of anti-CD94 Ab (Purified Mouse Anti-Human CD94, clone HP-3D9, BD Pharmingen) diluted in PBS with BSA 0.1%.
Pellet the cells by centrifugation (1 min at 2500 g). Resuspend cell pellets with 50 μl of CD94 Blocking cocktail and incubate for 30min at RT.
Prepare 2× Staining cocktail containing 20 μg/ml of CMV-peptide/HLA-E tetramers PE-labeled, anti-CD3 Ab Alexa-488-labeled (diluted 1:10) (Alexa Fluor 488 Mouse Anti-Human CD3, clone UCHT1, BD Pharmingen), anti-CD8 Ab APC-H7-labeled (diluted 1:25) (APC-H7 Mouse Anti-Human CD8, clone SK1, BD Pharmingen) and anti-γδTCR Ab APC-labeled (diluted 1:20) (APC Mouse Anti-Human TCR γδ, clone B1, BD Pharmingen) diluted in PBS with BSA 0.1%.
Add 50 μL of 2× Staining cocktail per well and incubate for 1 H at 4° C.
Wash cells twice: pellet the cells by centrifugation (1 min at 2500 g) and resuspend the cell pellet with 200 μl per well of PBS with BSA 0.1%.
Pellet the cells by centrifugation (1 min at 2500 g) and resuspend the cell pellet with 200 μl per well of PBS.
Analyze stained cell samples by Flow Cytometry.

Results

Frequency and Phenotypic Characteristics of HLA-E-Reactive CD8 T Cells Isolated From Peripheral Blood of a Cytomegalovirus-Seropositive Kidney-Transplant Patient

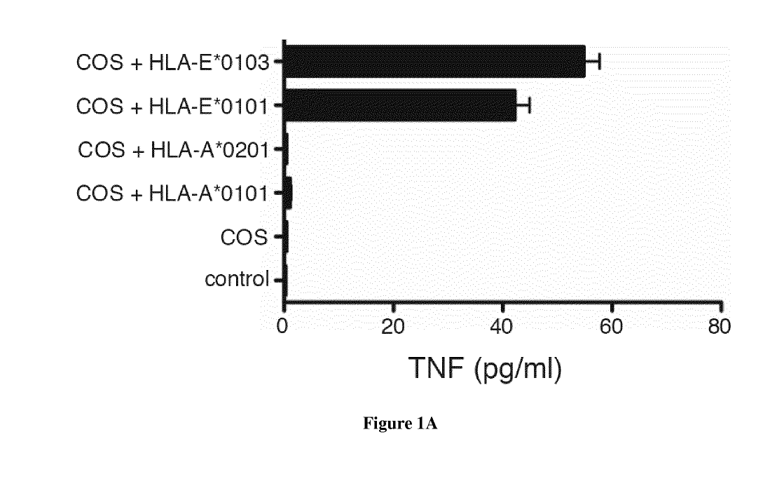

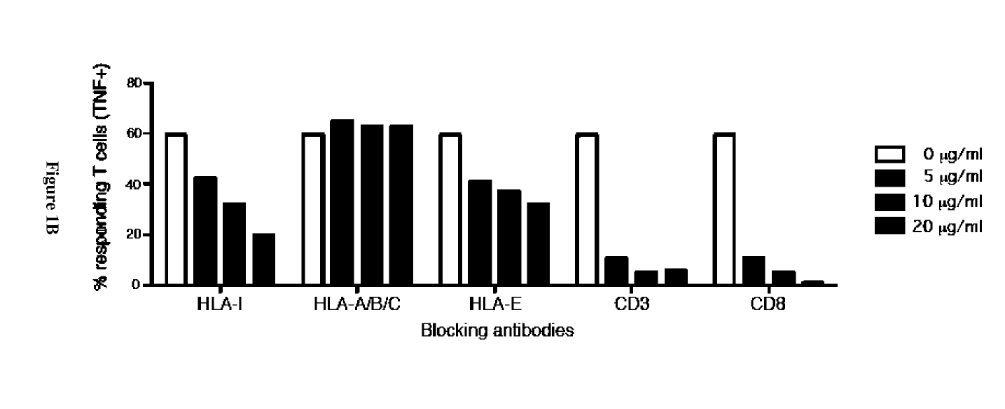

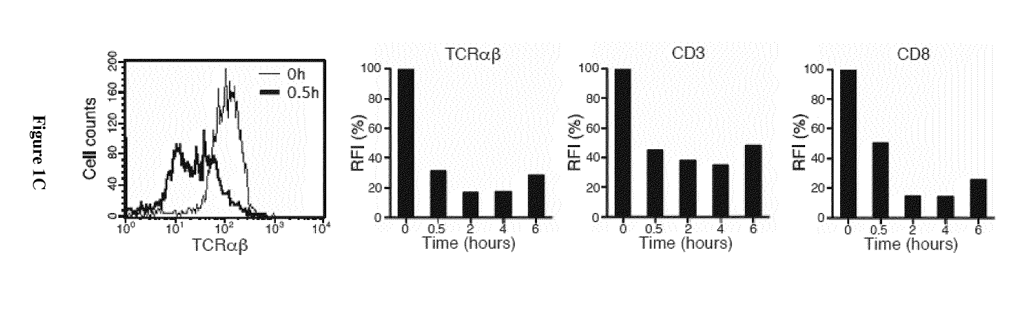

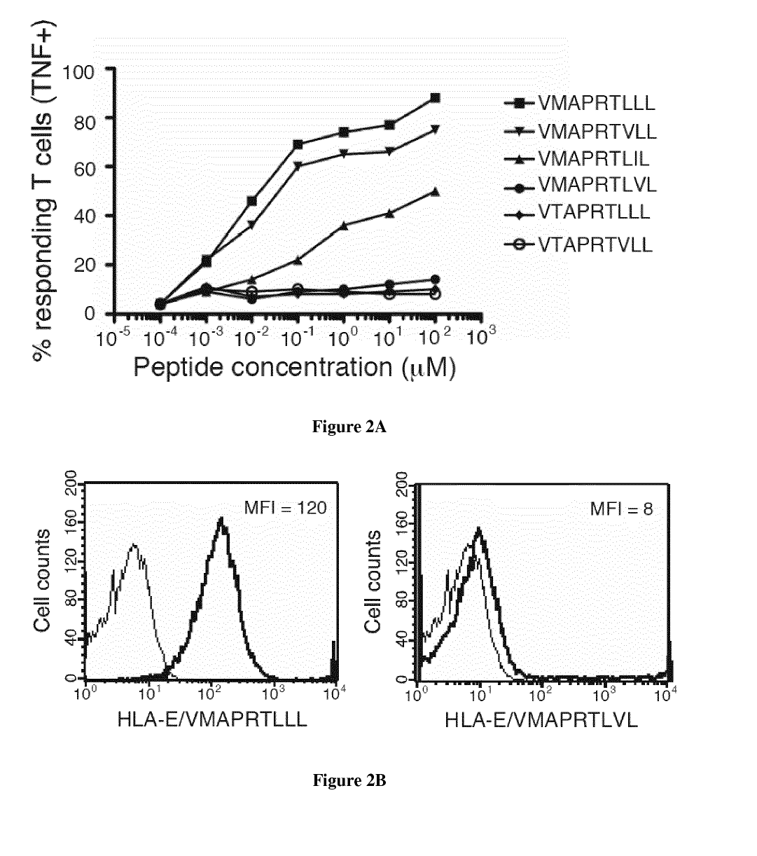

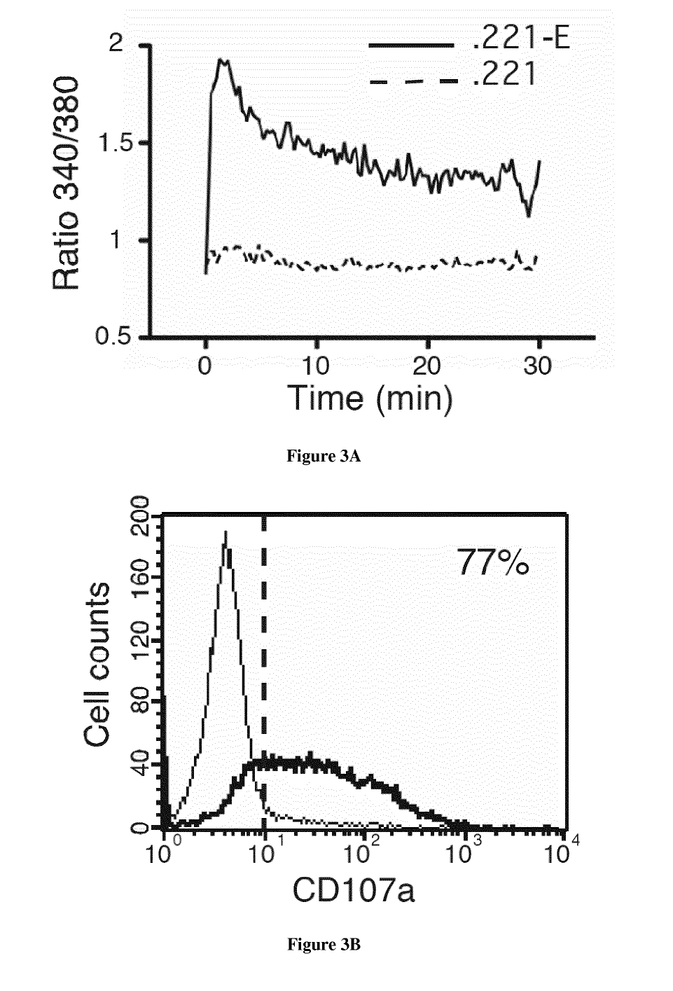

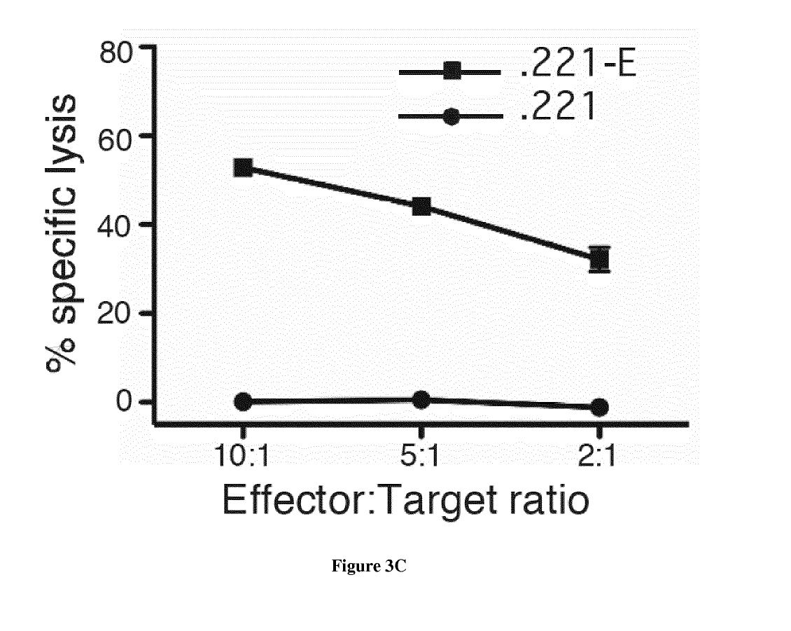

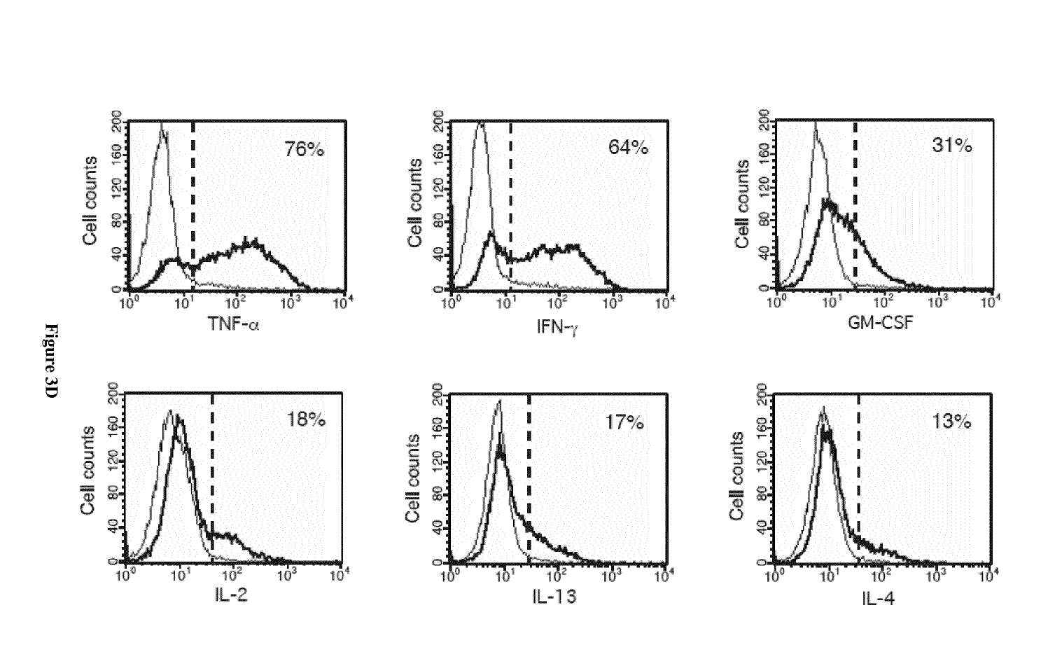

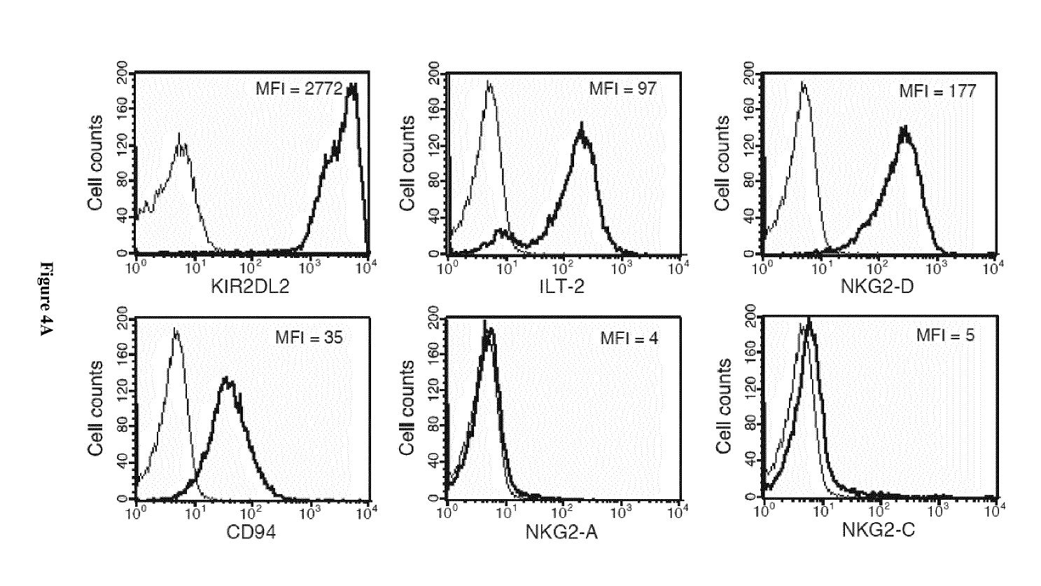

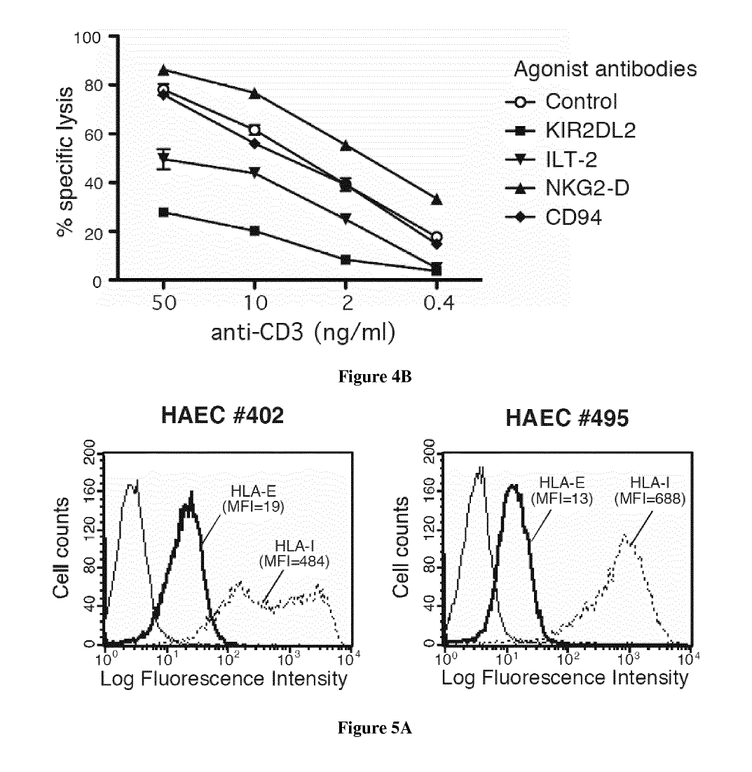

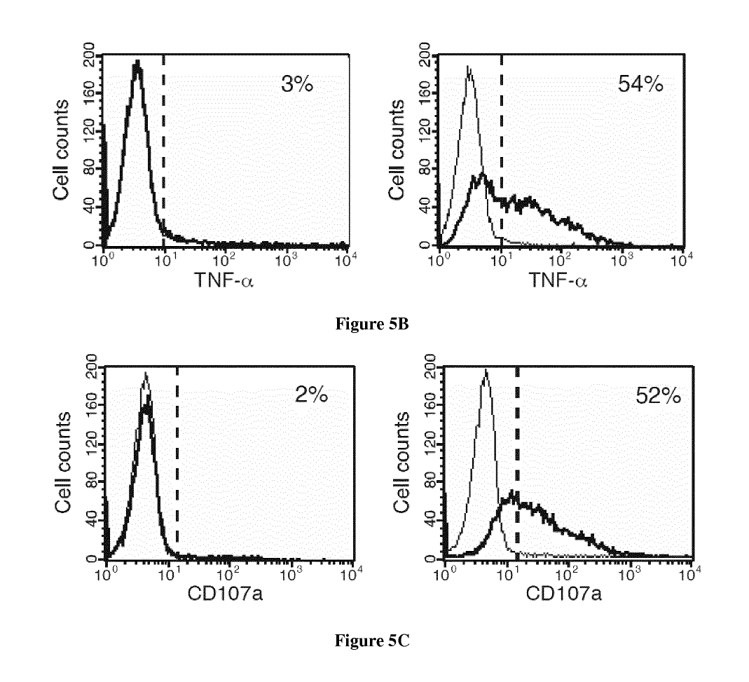

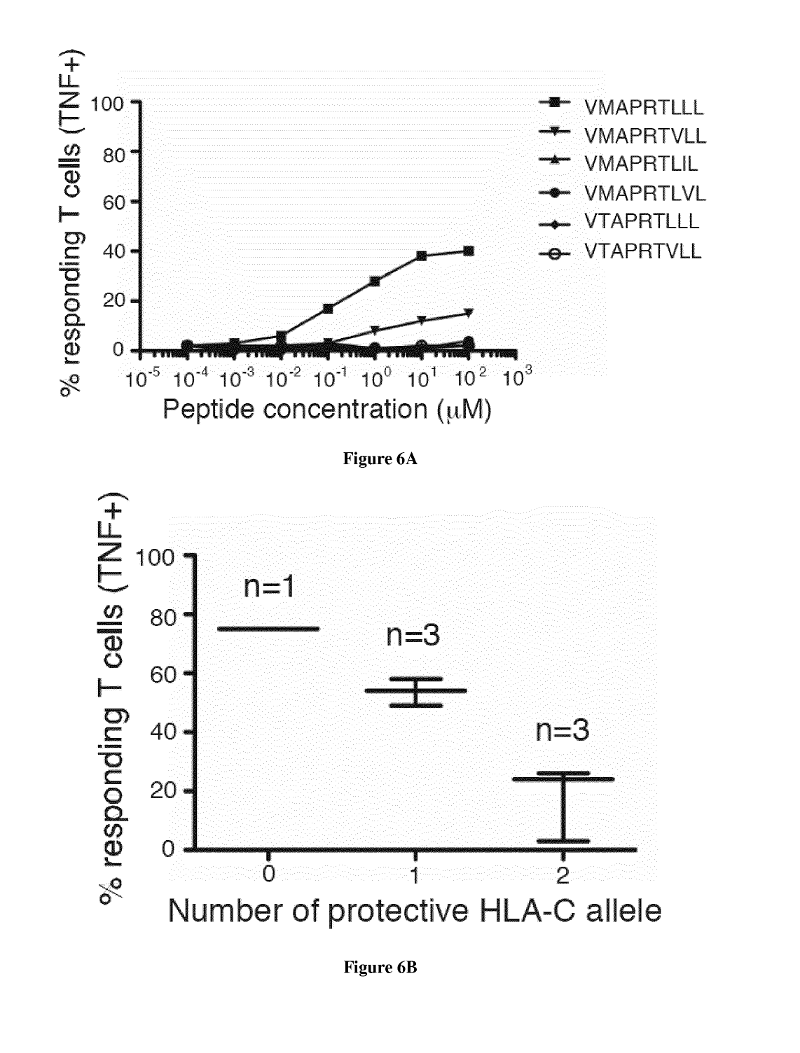

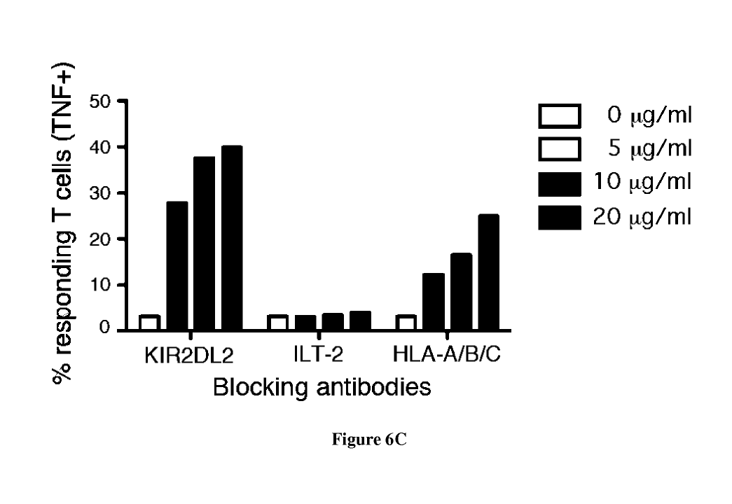

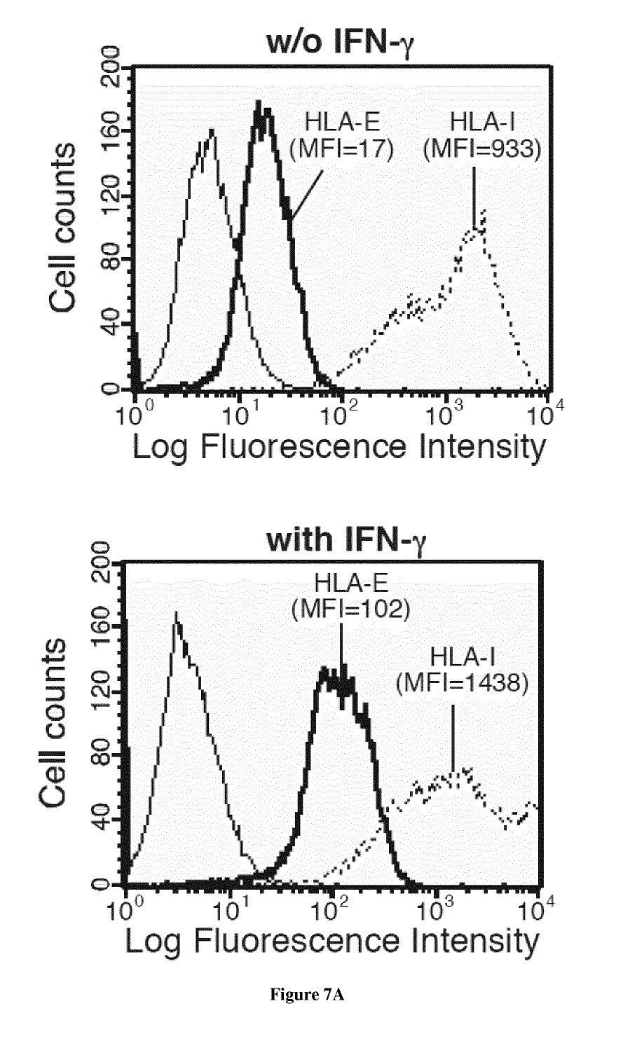

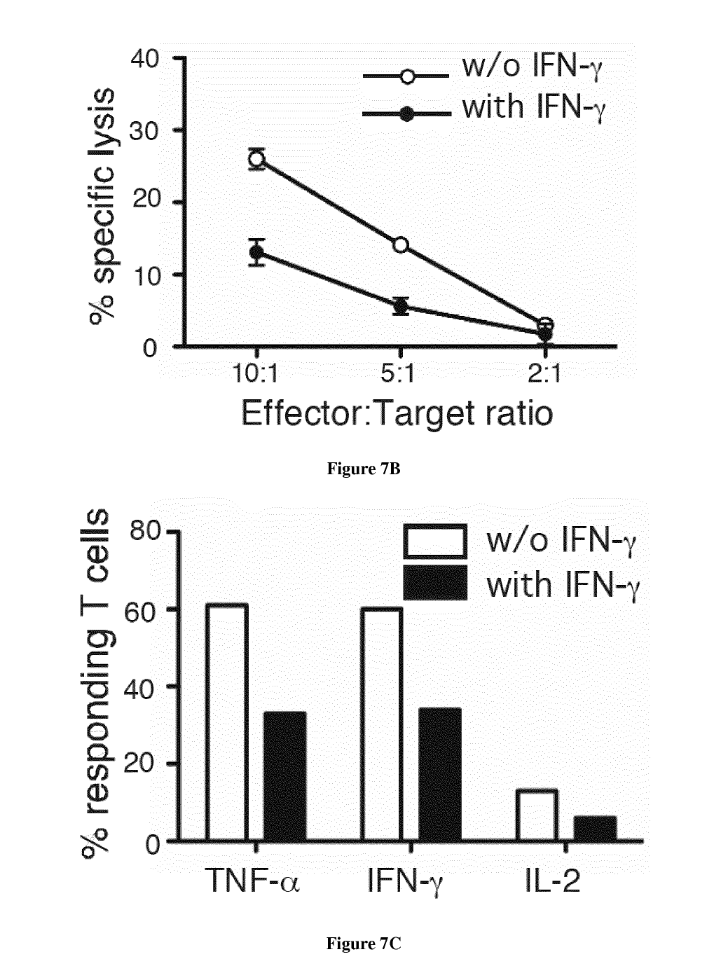

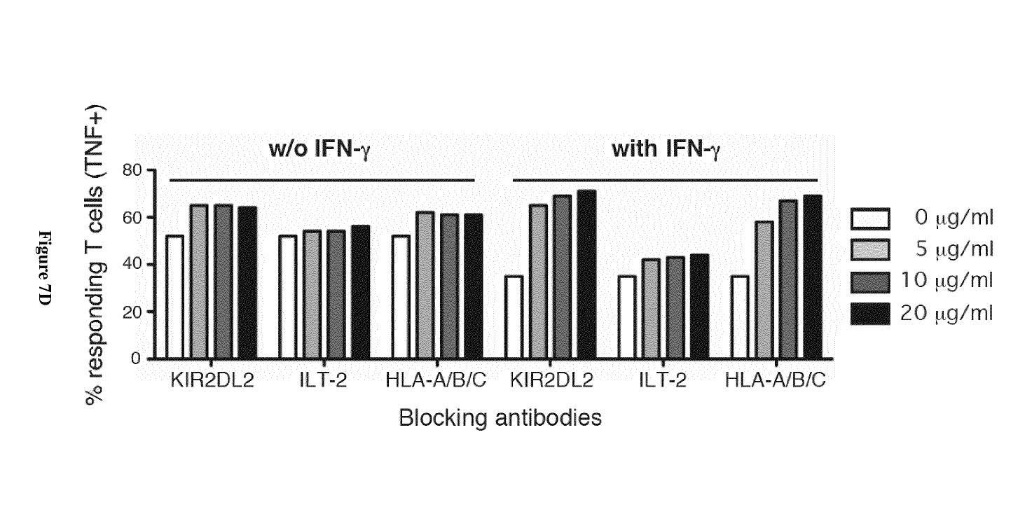

Investigations of a cohort of renal transplant recipients(12) allowed us to identify an HLA-E-reactive CD8⁺ T cell population in PBL of a kidney transplant recipient with an active CMV infection. This HLA-E-restricted response was not observed on blood samples harvested before CMV infection (at one month post-transplantation) but appeared correlated with CMV infection 2 years post-transplantation, in association with a T cell response to pp65₄₉₅₋₅₀₃/A*0201 HCMV epitope. As shown in FIG. 1A, recipient PBL activity, assessed by TNF-α production, was observed against COS-7 cells transfected with either HLA-E*0101 or HLA-E*0103 alleles whereas no response was observed with other HLA-I alleles tested. The HLA-E-reactive population was enriched and cloned. All the CD8 T cell clones derived (n=9) were HLA-E-reactive and characterized by the homogeneous expression of the TCRVβ22 (unpublished data). Notably, TCRVβ22⁺ cells represent a sizable fraction (6%) of freshly isolated recipient PBMC, comprising 14% of CD8⁺CD3⁺ T cells (unpublished data). This monoclonal population, thereafter named MART.22, is characterized by CD8αβ⁺CD62L⁻CCR7⁻CD27⁻CD28^{+/-}CD45RA^{lo}CD45RO^{hi}CD57⁻ surface phenotype (unpublished data), suggesting that MART.22 belongs to the effector-memory cell compartment(35). Moreover, MART.22 expresses CD56 consistent with the phenotype of HLA-E-restricted NK-CTL previously reported by the group of L. Moretta(13).

Requirement of Co-Engagement of TCR and CD8 for HLA-E-reactive CD8 T Cells

To further characterize MART.22, we used the 721.221 B-EBV cell line (.221), lacking classical HLA class I molecules and HLA-G expression, and the 721.221-AEH cell line (.221-E), which has been stably transfected with the cDNA encoding HLA-E*0101 together with the leader sequence peptide from HLA-B*08, that is required for HLA-E cell surface expression and stabilization(17). The transfected .221-E cell line, that consistently expresses high levels of HLA-E, induced strong activation of MART.22, as assessed by TNF production (59% of TNF-α producing T cells) (FIG. 1B, white histogram), whereas .221 cells were not recognized (FIG. 3).

To assess the contribution of T cell receptor and HLA-E interaction to target cell recognition, we performed antibody blocking experiments and TCR down-regulation analysis. A dose-dependent reduction of TNF-α producing T cells was observed in the presence of anti-CD3 (until 5% vs 59%), anti-HLA-I molecules (W6/32, until 20% vs 59%) or anti-HLA-E molecules (3D12, until 2% vs 59%) blocking antibodies (FIG. 1B). By contrast blocking antibody specific for HLA-A/B/C molecules (G46-2.6) had no inhibitory effect on this process. TCR implication was also confirmed by the significant down-regulation of surface CD3/TCR complex after MART.22 stimulation with 221-E cells (FIG. 1C). Furthermore, using the same approaches, we showed the high degree of CD8 dependency of MART.22 (FIGS. 1B-C). Together, these data confirm HLA-E restriction of MART.22 and unveil its strong CD8 dependency.

Peptide specificity of HLA-E-restricted CD8 T cells Next, to investigate MART.22 peptide specificity, we test its ability to recognize .221 cells exogenously loaded with six HLA-E-restricted synthetic peptides (Table I). This peptide set included the three previously described peptides derived from the UL40 protein of different human CMV strains(36, 37) and the peptides derived from the majority of HLA-I leader sequences, including autologous HLA-I from the transplant recipient. We found that MART.22 recognized .221 cells pulsed with 3 out of 6 peptides tested (FIG. 2A). The VMAPRTLLL peptide was recognized with the highest avidity (EC50 at 1×10⁻² μM). This peptide is derived from both the UL40 of the clinically isolate CMV 3C strain(36) and the leader sequence of various allogeneic HLA-A and HLA-C molecules. MART.22 also recognized with high avidity the VMAPRTVLL peptide (EC50 at 2×10⁻² 1 μM), which is derived from the leader sequence of various allogeneic HLA-B, including the HLA-B*08, molecules, thus providing explanation for the recognition of .221-E cells expressing HLA-B*08 leader sequence. MART.22 also recognized, albeit to a lesser extent (EC50 at 4×10⁻² μM), the VMAPRTLIL peptide that derived from the UL40 of the laboratory CMV AD169 strain(36, 37). This latter result was unexpected as this peptide also derives from the leader sequence of various HLA-C molecules, including the two autologous HLA-C alleles of the patient (ie HLA-Cw*1402 and -Cw*0501). The three other tested peptides (VTAPRTLLL, VTAPRTVLL and VMAPRTLVL) were not recognized at all, pinpointing to the importance of a methionine and of a leucine or an isoleucine at position 2 and 8 respectively to allow peptide recognition. To further substantiate our data on MART.22 peptide specificity, we used HLA-E*0101 tetramers refolded with either VMAPRTLLL or VMAPRTLVL peptides. As expected, FIG. 2B shows the ability of MART.22 to bind HLA-E/VMAPRTLLL tetramers whereas no significant binding was observed with tetramers refolded with the unrecognized VMAPRTLVL peptide.

Functional Characteristics of HLA-E-Restricted CD8 T Cells

Functional characterization of MART.22 was assessed using .221-E stimulating cells. As shown in FIG. 3A, incubation with .221-E cells triggered a strong and rapid elevation in intracellular free calcium (Ca²⁺) concentration within MART.22 while no significant Ca²⁺ signal was detected when untransfected .221 cells were used. With regard to its potential ability to develop lytic response, incubation with .221-E cells induced MART.22 degranulation as demonstrated by the high CD107a surface mobilization (77% of CD107a positive T cells) and perforin/granzyme production (FIG. 3B and unpublished data). This leads to the lysis of .221-E cells as assessed with a standard ⁵¹Cr release assay (FIG. 3C). As shown in FIG. 3D, MART.22 was also found to produce high levels of TNF-α (78% of producing cells), IFN-γ (64%) and to a lower extent GM-CSF (31%), IL-2 (18%), IL-13 (17%) and IL-4 (13%). Conversely, no production of IL-5, IL-17F, IL-21, IL-22 or TGF-β was detected (unpublished data). These data emphasize the strong granzyme-dependent cytolytic and TNF-α/IFN-γ secretion capacities of MART.22.

Regulation of HLA-E-Restricted CD8 T Cells Activity by NKR

As previous studies on HLA-E-restricted NK-CTL reported surface expression of HLA class I-specific inhibitory NK receptors (NKR), we investigated NKR expression on MART.22 (FIG. 4A). MART.22 was strongly stained by the GL183 antibody, which recognizes KIR2DS2, KIR2DL2 and KIR2DL3. The combined use of KIR-specific mAbs(38) allowed us to identify the inhibitory KIR2DL2 as the KIR expressed by MART.22 (unpublished data). Surface expression of ILT-2, NKG2-D and CD94 were also observed. Surprisingly, CD94 expression was not associated with NKG2-A or NKG2-C surface expression. In order to address the functionality of these receptors, we analyzed, in a redirected lysis assay, the ability of anti-NKR mAbs to modulate MART.22 TCR dependent lysis. As shown in FIG. 4B, anti-CD3 mAb induced cytolytic activity was strongly inhibited by the addition of anti-KIR2DL2 mAb. Lysis was also inhibited, although to a lesser extent, by the addition of anti-ILT-2 mAb while it was slightly increased in presence of anti-NKG2-D mAb. However, addition of anti-CD94 mAb did not affect the lysis efficiency, clearly indicating the non-functionality of the CD94 receptor expressed by MART.22. Taken together, our data clearly indicate that the activity of HLA-E-restricted T cells can be modulated by competing positive or negative signals transduced by NKR, with especially efficient inhibition through KIR2DL2 ligation. Interestingly, autologous MART.22 HLA-C molecules (HLA-Cw*0501 and *1402) are ligands for the KIR2DL2 receptor(39). Since these HLA-C molecules also provide a recognized HLA-E-bound peptide (FIG. 2A and Table II), this allowed us to hypothesize that inhibitory KIR2DL2 expression by MART.22 dampens its detrimental auto-reactivity against healthy (not CMV infected) autologous cells through ligation of autologous protective HLA-C molecules. Accordingly, when incubated in the presence of anti-KIR2DL2/DS2/DL3 or HLA-A/B/C blocking Abs, MART.22 developed fratricide response (Suplemental FIG. 2).

HLA-E-restricted CD8 T Cells Reactivity against Allogeneic Endothelial Cells

Since we demonstrated that peptides derived from both CMV-UL40 and allogeneic HLA-I molecules can be recognized by MART.22 in an HLA-E-restricted fashion, we asked whether MART.22 could also recognize and damage allogeneic endothelial cells and therefore represent a risk factor for allograft outcome. To this end, primary human arterial endothelial cell (HAEC) cultures, isolated from kidney transplant donors were tested in vitro for their capacity to activate MART.22. HLA-I typing of the seven endothelial cell cultures tested as well as their capacity to provide recognized peptides or to interact with KIR2DL2 are documented in Table II. All EC cultures expressed HLA-I molecules carrying peptides potentially recognized in the HLA-E context. The CMV serologic status of EC donors is also indicated. While surface HLA-E staining levels were similar on all EC cultures tested (FIG. 5A and unpublished data), six out of seven EC cultures induced efficient cytokine responses of MART.22, as illustrated by TNF-α production (from 24% to 75% of T cells) (FIG. 5B and Table II). Moreover, MART.22 develops cytolytic responses against recognized endothelial cells, as assessed by CD107a surface expression (from 8% to 68% of T cells) (FIG. 5C and Table II). In accordance with recognition of both allelic forms of HLA-E by MART.22 (FIG. 1A), endothelial cells are recognized independently of their HLA-E haplotype and with no correlation to CMV infection (mean value, 42% of TNF producing T cells for CMV negative versus 41% for CMV positive patients), suggesting the direct recognition of allogeneic HLA-I derived peptides in an HLA-E-restricted fashion. Thus, HLA-E-restricted T cells could represent a risk factor for allograft outcome through recognition of allogeneic graft endothelial cells.

Tight Regulation of HLA-E-Restricted CD8 T Cells Alloreactivity by KIR2DL2

As mentioned above, in an unexpected way, one EC culture (HAEC#402), with no apparent defect in surface HLA-E expression levels, was not recognized by MART.22 (FIG. 5A). To ascertain this was not the consequence of the specific lack of expression of HLA-I molecules encoding recognized peptides, we investigated whether incubation with the two best-recognized synthetic peptides could render these endothelial cells more susceptible to recognition by MART.22. As shown on FIG. 6A, pulsing of the otherwise resistant HAEC#402 with VMAPRTLLL and VMAPRTVLL induced TNF-α production by MART.22 but only with saturating amounts of peptides (respectively 40% and 18% of TNF secreting T cells when HAEC#402 were loaded with 10² μM of peptides). Similar results were obtained with another poorly recognized EC culture (unpublished data), suggesting another mechanism conferring resistance to recognition. As we showed that MART.22 reactivity is strongly regulated by the inhibitory KIR2DL2, we investigated whether HAEC suboptimal recognition was indeed the consequence of the expression of protective HLA-C molecules (ie KIR2DL2 ligands)(39-41). Interestingly, HLA-C haplotype crucially influence the MART.22 alloreactivity: endothelial cells possessing two appropriate HLA-C alleles (HAEC#116, #337 and #402) are less recognized (mean value, 18% of TNF producing T cells) than those bearing only one (HAEC#112, #331 and #495, 54% of TNF producing T cells) or no (HAEC#323, 75%) (FIG. 6B). This was confirmed by assessing the effect of blocking antibodies on endothelial cells recognition by MART.22. As shown on FIG. 6C, addition of KIR2DL2-blocking Abs and, to a lesser extent, of anti-HLA-A/B/C Abs efficiently restore the HAEC#402 recognition by MART.22 in a dose dependent manner (up to 40% and 25% respectively), whereas addition of blocking Ab to ILT-2 had no significant effect. These results underline the tight regulation of HLA-E-restricted allo-reactivity by KIR2DL2 receptors through their recognition of HLA-C molecules expressed on target cells.

Effect of IFN-γ Treatment on Endothelial Cells Recognition by HLA-E-Restricted CD8 T Cells

Chronic CMV infections result in recruitment of inflammatory cells and mediators such as chemokines and cytokines including IFN-γ(4). So, we analyzed the impact of IFN-γ treatment of EC cultures on their recognition by MART.22. As we previously reported(29), IFN-γ treatment enhances both HLA-E and total HLA-I surface expression on endothelial cells (FIG. 7A and unpublished data). However, IFN-γ treatment of endothelial cells resulted in decreased MART.22 mediated lysis and cytokine production (FIGS. 7B-C). The percentage of TNF-αproducing T cells upon stimulation with the HAEC#495 fell from 61% to 33% after IFN-γ treatment. Experiments performed with a less recognized EC culture show that MART.22 reactivity against IFN-γ treated HAEC#116 was completely abolished (unpublished data). To investigate whether the inhibitory effect of IFN-γ treatment was the consequence of an increased expression of inhibitory NKR ligands by endothelial cells, we performed antibody blocking experiments. First, anti-KIR2DL2 and anti-ILT-2 antibodies had little or no effect on recognition of the untreated HAEC#495 culture. In contrast, these antibodies, especially the anti-KIR2DL2 mAb, improved in a dose dependent manner the recognition of IFN-γ treated endothelial cells (71% vs 36% of TNF-α producing T cells for the maximal dose of anti-KIR2DL2 Ab) (FIG. 7D). In the same way, mAb directed against classical HLA-I molecules, which are ligands of both KIR2DL2 and ILT-2, greatly enhanced recognition of IFN-γ treated endothelial cells recognition (69% vs 36% of TNF-α producing T cells for the maximal dose of Ab). Taken together, these data underline the crucial role of inhibitory NKR ligands which expression on EC is a determining factor for HLA-E-restricted T cells reactivity.

Discussion:

In conclusion, this study demonstrates for the first time the ability of CMV-committed HLA-E-restricted T cells from transplant recipient to recognize and lyse allogeneic endothelial cells thereby emphasizing their potential detrimental alloreactivity upon solid organ transplantation.

A function for HLA-E as a restricting element for the TCR of αβ T cells has been clearly established(20) and therefore can play a role in the adaptive immune response in addition to its well-known regulation of innate immunity(42, 43). The HLA-E-restricted CD8 αβ T cell population described in this study appears in association with a T cell response to classical HLA I-restricted HCMV epitope (pp65/A*02) in the blood of a kidney transplant recipient with an active CMV infection. Thus, HLA-E-restricted T cells may be induced in vivo in recipient patients as a consequence of CMV infection or reactivation, suggesting their possible role in the immune adaptative response to CMV. Various CMV proteins inhibit MHC class Ia surface expression impeding the control mediated by conventional (i.e. MHC class Ia-restricted) CD8 T cells(44, 45). Therefore, the capacity of CMV, through the expression of UL40, to supply HLA-E-binding peptides allowing increase of HLA-E surface expression in infected cells(37), strengthen that CMV-commited HLA-E-restricted T cells may have a particular relevance in the immune defense against CMV.

In accordance with previous studies showing that CMV-committed HLA-E-restricted T cells represent a pauciclonal population comprising a sizable fraction of CD8 αβ T cells in CMV-seropositive patients(15, 46), the population described in this study expresses homogeneously a given TCR owing to its monoclonal origin and constitutes a significant component of peripheral blood mononuclear cells (14% of CD8⁺CD3⁺T cells). Moreover, we showed that this population has phenotypic characteristics of effector-memory lymphocytes and displays strong granzyme-dependent cytolytic and TNF-α/IFN-γ secretion capacities, suggesting that they could play a relevant role in the control of CMV infection.

As three different HLA-E-binding HCMV-UL40-derived peptides have been previously described, we investigated the specificity of our HLA-E-restricted T cells. Previous studies from the group of L. Moretta have characterized HLA-E-restricted T cells reacting against peptides (i.e. VMAPRTLIL and VMAPRTLVL) derived from the UL40 of 2 HCMV laboratory strains (Toledo and AD169 strains)(15). The HLA-E-restricted T cell population described here reacts against the additional UL40 derived-peptide, VMAPRTLLL, that has been shown to derive from the clinical isolate HCMV 3C strain.

Because recognized peptides also derived from the leader sequences of numerous allogeneic HLA-I alleles, CMV-committed HLA-E-restricted T cells have potential to mediate allograft rejection through direct recognition of allogeneic HLA-I derived-peptides presented by HLA-E on graft cells. In a previous study, we showed that HLA-E protein expression in normal human organs is mainly restricted to endothelial cells and leucocytes(29). Hence, owing to the crucial role of endothelial cells in allo-αntigen presentation to T cells(8) and to the HCMV tropism for endothelial cells(5, 7), we investigated whether HLA-E-restricted T cells could recognize primary endothelial cells cultures, isolated from kidney allografts. We clearly demonstrate that CMV-committed HLA-E-restricted CD8 T cells can efficiently recognized and killed allogeneic endothelial cells in vitro, independently of their HLA-E allotype. Therefore, because immunosuppressed transplant patients are particularly prone to CMV infection, we can speculate that in the context of both CMV reactivation or primary infections, while these T cells have potential to contribute to infection control, they may also directly recognize allogeneic graft endothelial cells and thereby contribute to allograft rejection.

As suggested by previous studies, we clearly demonstrated that CMV-committed HLA-E-restricted T cell allo-reactivity is tightly regulated by NK receptors(47). We first showed surface expression of KIR2DL2, ILT-2, NKG2D and CD94 receptors by MART.22. Surprisingly, CD94 surface expression was not associated with that of NKG2-A or NKG2-C molecules and did not allow interaction with HLA-E tetramer refolded with HLA-A2 peptide, suggesting the expression of CD94 homodimers as previously described(48). Finally, we demonstrated the non-functionality of this receptor. All the other expressed NK receptors were found to be functional, with a predominant role in preventing target cell recognition for the highly expressed inhibitory KIR2DL2 through ligation of appropriated (protective) HLA-C molecules(39). The expression of KIR2DL2 appears to constitute a safety mechanism avoiding harmful autoreactivity through the ligation of protective autologous HLA-C molecules. As a consequence, the ability of HLA-E-restricted T cells to mediate alloreactivity against endothelial cells was crucially impacted by the expression of protective HLA-C alleles. Thus, allogeneic endothelial cells that express protective HLA-C molecules, or that were pre-treated with INF-γ, were less recognized by HLA-E-restricted T cells, unless specific blocking antibodies (i.e. anti-KIR2DL2 or anti-HLA-A/B/C) were added to the cultures. This underlines the crucial impact of HLA-C haplotype of target cells on their ability to trigger, or not, an allogeneic HLA-E-restricted T cell response. Therefore, HLA-C haplotypes that are still underestimated in transplantation setting should be reconsidered and taken into account(49, 50).

In conclusion, we demonstrated, for the first time, that immune control of CMV infection in transplanted patient trigger HLA-E-restricted T cells that can mediate detrimental vascularized allograft rejection via endothelial cells lysis. Therefore, CMV-committed HLA-E restricted T cells could account for the well-established association between CMV-infection and accelerated allograft rejection. As HLA-E is also expressed in leucocytes, the involvement of HLA-E-restricted T cells in the immunological response following allogeneic hematopoietic stem cell transplantation should also be addressed, as it has been suggested by studies using transgenic mice(31). Moreover, we provided strong evidence that HLA-C/NKR mismatch is a key player in HLA-E-restricted T cells alloreactivity. Thus, graft organ HLA-C haplotypes may impact on CMV-committed HLA-E-restricted T cells capacity to mediate allograft rejection. Hence, a deeper evaluation of the frequency and the role of CMV-committed HLA-E-restricted T cells in transplantation and of the impact of HLA-C haplotype on their alloreactivity, may determine whether this indeed represents an additional risk factor following solid organ transplantation.

TABLE I
Leader sequence peptides derived from HCMV-UL40/HLA-I
molecules and their recognition by HLA-E-restricted T cell clone

| Leader sequence | | MART.22 |
|---|---|---|
| peptide₃₋₁₁ | HLA class I allotypes | Reactivity^{a} |
| VMAPRTLVL^{b, c} | HLA-A*02, -A*23, -A*24, -A*25, -A*26, -A*3402, -A*43, | − |
| | -A*66 and -A*69, | |
| VMAPRTLLL^{b} | HLA-A*01, -A*03, -A*11, -A*29, -A*30, -A*31, -A*32, | +++ |
| | -A*33, -A*36 -A*74, -Cw*2 and -Cw*15 | |
| VMAPRTLIL^{b, c} | HLA-Cw*01, -Cw*03, -Cw*0401, -Cw*05, -Cw*06, | + |
| | -Cw*0801-03, -Cw*12, -Cw*14, -Cw*16 and -Cw*1702 | |
| VMAPRTVLL | HLA-B*07, -B*08, -B*14, -B*38, -B*39, -B*42, -B*67, | ++ |
| | -B*73 and -B*81 | |
| VTAPRTLLL | HLA-B*13, -B*18, -B*27, -B*3542, -B*37, -B*40, -B*44, | − |
| | -B*47, -B*54, -B*56, -B*58, -B*59, -B*82 and -B*83 | |
| VTAPRTVLL | HLA-B*15, -B*35, -B*40, -B*41, -B*4418, -B*45, -B*49, | − |
| | -B*50, -B*51, -B*52, -B*57 and -B*78 | |

Autologous HLA class I alleles of the transplant recipient are indicated in bold.
^{a}MART.22 HLA-E-restricted T cell clone activity in response to .221 cells pulsed with different peptides (see FIG. 3)
^{b}These peptides are identical to peptides contained in the UL40 ORF from various CMV strains.
^{c}These pepides have previously been described for their ability to trigger HLA-E restricted CD8 T cell responses.

TABLE II
Characteristics of endothelial cells (HLA class I allotypes
and HCMV serologic status of donors) and their recognition by HLA-E-
restricted T cell clone

| | | | | | HCMV | MART.22 | |
|---|---|---|---|---|---|---|---|
| | HLA-Ia allotypes | | | HLA-E | Sero- | Reactivity^{a} | |
| HAEC | HLA-A | HLA-B | HLA-Cw | allotypes | positivity | TNF-α | CD 107^{a} |

| | | | | | | | | | | | |
|---|---|---|---|---|---|---|---|---|---|---|---|
| #112 | *0201 | *2402 | *1801 | *5101 | *0202 | *0701^{b} | *0103 | *0103 | + | 49% | 32% |
| #116 | *0201 | *2902 | *3501 | *4402 | *0401^{c} | *0501^{b} | *0101 | *0101 | + | 24% | 8% |
| #323 | *0301 | *2402 | *4701 | *5001 | *0602 | *0602 | ND | ND | − | 75% | 68% |
| #331 | *0301 | *3201 | *0702 | *3701 | *0602 | *0702^{b} | *0101 | *0103 | − | 58% | 41% |
| #337 | *2402 | *3101 | *3501 | *4001 | *0401^{c} | *0304^{b} | *0103 | *0103 | − | 26% | 10% |
| #402 | *2301 | *2902 | *4403 | *5801 | *0701^{b} | *1601^{b} | *0101 | *0103 | − | 3% | 4% |
| #495 | *0101 | *0201 | *4101 | *4402 | *0501^{b} | *1701 | ND | ND | + | 54% | 52% |

HLA-Ia alleles susceptible to provide peptides recognized by HLA-E-restricted T cell clone are indicated in bold.
^{a}HLA-E-restricted T cell clone activity in response to endothelial cells (see FIG. 6)
^{b}HLA-C allotypes carrying the C1 epitope that are susceptible to bind to KIR2DL2 receptor
^{c}HLA-Cw0401 allotype that has been shown to interact with KIR2DL2 receptor

### REFERENCES

Throughout this application, various references describe the state of the art to which this invention pertains. The disclosures of these references are hereby incorporated by reference into the present disclosure.
1. Husain, S., C. E. Pietrangeli, and A. Zeevi. 2009. Delayed onset CMV disease in solid organ transplant recipients. Transpl Immunol 21:1-9.
2. Rowshani, A. T., F. J. Bemelman, E. M. van Leeuwen, R. A. van Lier, and I. J. ten Berge. 2005. Clinical and immunologic aspects of cytomegalovirus infection in solid organ transplant recipients. Transplantation 79:381-386.
3. Fisher, R. A. 2009. Cytomegalovirus infection and disease in the new era of immunosuppression following solid organ transplantation. Transpl Infect Dis 11:195-202.
4. Streblow, D. N., S. L. Orloff, and J. A. Nelson. 2007. Acceleration of allograft failure by cytomegalovirus. Curr Opin Immunol 19:577-582.
5. Adler, B., and C. Sinzger. 2009. Endothelial cells in human cytomegalovirus infection: one host cell out of many or a crucial target for virus spread? Thromb Haemost 102:1057-1063.
6. Guetta, E., E. M. Scarpati, and P. E. DiCorleto. 2001. Effect of cytomegalovirus immediate early gene products on endothelial cell gene activity. Cardiovasc Res 50:538-546.
7. Sacher, T., C. A. Mohr, A. Weyn, C. Schlichting, U. H. Koszinowski, and Z. Ruzsics. The role of cell types in cytomegalovirus infection in vivo. Eur J Cell Biol.
8. Al-Lamki, R. S., J. R. Bradley, and J. S. Pober. 2008. Endothelial cells in allograft rejection. Transplantation 86:1340-1348.
9. Amir, A. L., L. J. D'Orsogna, D. L. Roelen, M. M. van Loenen, R. S. Hagedoorn, R. de Boer, M. A. van der Hoorn, M. G. Kester, Doxiadis, II, J. H. Falkenburg, F. H. Claas, and M. H. Heemskerk. Allo-HLA reactivity of virus-specific memory T cells is common. Blood 115:3146-3157.
10. D′Orsogna, L. J., D. L. Roelen, Doxiadis, II, and F. H. Claas. Alloreactivity from human viral specific memory T-cells. Transpl Immunol 23:149-155.
11. Gamadia, L. E., E. B. Remmerswaal, S. Surachno, N. M. Lardy, P. M. Wertheim-van Dillen, R. A. van Lier, and I. J. ten Berge. 2004. Cross-reactivity of cytomegalovirus-specific CD8+ T cells to allo-major histocompatibility complex class I molecules. Transplantation 77:1879-1885.
12. Morice, A., B. Charreau, B. Neveu, S. Brouard, J. P. Soulillou, M. Bonneville, E. Houssaint, and N. Degauque. Cross-reactivity of herpesvirus-specific CD8 T cell lines toward allogeneic class I MHC molecules. PLoS One 5:e12120.
13. Pietra, G., C. Romagnani, M. Falco, M. Vitale, R. Castriconi, D. Pende, E. Millo, S. Anfossi, R. Biassoni, L. Moretta, and M. C. Mingari. 2001. The analysis of the natural killer-like activity of human cytolytic T lymphocytes revealed HLA-E as a novel target for TCR alpha/beta-mediated recognition. Eur J Immunol 31:3687-3693.
14. Pietra, G., C. Romagnani, C. Manzini, L. Moretta, and M. C. Mingari. The emerging role of HLA-E-restricted CD8+ T lymphocytes in the adaptive immune response to pathogens and tumors. J Biomed Biotechnol 2010:907092.
15. Pietra, G., C. Romagnani, P. Mazzarino, M. Falco, E. Millo, A. Moretta, L. Moretta, and M. C. Mingari. 2003. HLA-E-restricted recognition of cytomegalovirus-derived peptides by human CD8+ cytolytic T lymphocytes. Proc Natl Acad Sci USA 100:10896-10901.
16. Strong, R. K., M. A. Holmes, P. Li, L. Braun, N. Lee, and D. E. Geraghty. 2003. HLA-E allelic variants. Correlating differential expression, peptide affinities, crystal structures, and thermal stabilities. J Biol Chem 278:5082-5090.
17. Lee, N., D. R. Goodlett, A. Ishitani, H. Marquardt, and D. E. Geraghty. 1998. HLA-E surface expression depends on binding of TAP-dependent peptides derived from certain HLA class I signal sequences. J Immunol 160:4951-4960.
18. Braud, V. M., D. S. Allan, C. A. O'Callaghan, K. Soderstrom, A. D'Andrea, G. S. Ogg, S. Lazetic, N. T. Young, J. I. Bell, J. H. Phillips, L.

L. Lanier, and A. J. McMichael. 1998. HLA-E binds to natural killer cell receptors CD94/NKG2A, B and C. Nature 391:795-799.
19. Lee, N., M. Llano, M. Carretero, A. Ishitani, F. Navarro, M. Lopez-Botet, and D. E. Geraghty. 1998. HLA-E is a major ligand for the natural killer inhibitory receptor CD94/NKG2A. Proc Natl Acad Sci U S A 95:5199-5204.
20. Hoare, H. L., L. C. Sullivan, G. Pietra, C. S. Clements, E. J. Lee, L. K. Ely, T. Beddoe, M. Falco, L. Kjer-Nielsen, H. H. Reid, J. McCluskey, L. Moretta, J. Rossjohn, and A. G. Brooks. 2006. Structural basis for a major histocompatibility complex class Ib-restricted T cell response. Nat Immunol 7:256-264.
21. van Hall, T., C. C. Oliveira, S. A. Joosten, and T. H. Ottenhoff. The other Janus face of Qa-1 and HLA-E: diverse peptide repertoires in times of stress. Microbes Infect 12:910-918.
22. Joosten, S. A., K. E. van Meijgaarden, P. C. van Weeren, F. Kazi, A. Geluk, N. D. Savage, J. W. Drijfhout, D. R. Flower, W. A. Hanekom, M. R. Klein, and T. H. Ottenhoff. Mycobacterium tuberculosis peptides presented by HLA-E molecules are targets for human CD8 T-cells with cytotoxic as well as regulatory activity. PLoS Pathog 6:e1000782.
23. Salerno-Goncalves, R., M. Fernandez-Vina, D. M. Lewinsohn, and M. B. Sztein. 2004. Identification of a human HLA-E-restricted CD8+ T cell subset in volunteers immunized with Salmonella enterica serovar Typhi strain Ty21a typhoid vaccine. J Immunol 173:5852-5862.
24. Garcia, P., M. Llano, A. B. de Heredia, C. B. Willberg, E. Caparros, P. Aparicio, V. M. Braud, and M. Lopez-Botet. 2002. Human T cell receptor-mediated recognition of HLA-E. Eur J Immunol 32:936-944.
25. Schulte, D., M. Vogel, B. Langhans, B. Kramer, C. Korner, H. D. Nischalke, V. Steinberg, M. Michalk, T. Berg, J. K. Rockstroh, T. Sauerbruch, U. Spengler, and J. Nattermann. 2009. The HLA-E(R)/HLA-E(R) genotype affects the natural course of hepatitis C virus (HCV) infection and is associated with HLA-E-restricted recognition of an HCV-derived peptide by interferon-gamma-secreting human CD8(+) T cells. J Infect Dis 200:1397-1401.
26. Sensi, M., G. Pietra, A. Molla, G. Nicolini, C. Vegetti, I. Bersani, E. Millo, E. Weiss, L. Moretta, M. C. Mingari, and A. Anichini. 2009. Peptides with dual binding specificity for HLA-A2 and HLA-E are encoded by alternatively spliced isoforms of the antioxidant enzyme peroxiredoxin 5. Int Immunol 21:257-268.
27. Jiang, H., S. M. Canfield, M. P. Gallagher, H. H. Jiang, Y. Jiang, Z. Zheng, and L. Chess. HLA-E-restricted regulatory CD8(+) T cells are involved in development and control of human autoimmune type 1 diabetes. J Clin Invest 120:3641-3650.
28. Oliveira, C. C., P. A. van Veelen, B. Querido, A. de Ru, M. Sluijter, S. Laban, J. W. Drijfhout, S. H. van der Burg, R. Offringa, and T. van Hall. The nonpolymorphic MHC Qa-1b mediates CD8+ T cell surveillance of antigen-processing defects. J Exp Med 207:207-221.
29. Coupel, S., A. Moreau, M. Hamidou, V. Horejsi, J. P. Soulillou, and B. Charreau. 2007. Expression and release of soluble HLA-E is an immunoregulatory feature of endothelial cell activation. Blood 109:2806-2814.
30. Choi, J., D. R. Enis, K. P. Koh, S. L. Shiao, and J. S. Pober. 2004. T lymphocyte-endothelial cell interactions. Annu Rev Immunol 22:683-709.
31. Pacasova, R., S. Martinozzi, H. J. Boulouis, M. Ulbrecht, J. C. Vieville, F. Sigaux, E. H. Weiss, and M. Pla. 1999. Cell-surface expression and alloantigenic function of a human nonclassical class I molecule (HLA-E) in transgenic mice. J Immunol 162:5190-5196.
32. Saulquin, X., C. Ibisch, M. A. Peyrat, E. Scotet, M. Hourmant, H. Vie, M. Bonneville, and E. Houssaint. 2000. A global appraisal of immunodominant CD8 T cell responses to Epstein-Barr virus and cytomegalovirus by bulk screening. Eur J Immunol 30:2531-2539.
33. Gervois, N., N. Labarriere, S. Le Guiner, M. C. Pandolfino, J. F. Fonteneau, Y. Guilloux, E. Diez, B. Dreno, and F. Jotereau. 2000. High avidity melanoma-reactive cytotoxic T lymphocytes are efficiently induced from peripheral blood lymphocytes on stimulation by peptide-pulsed melanoma cells. Clin Cancer Res 6:1459-1467.
34. Coupel, S., F. Leboeuf, G. Boulday, J. P. Soulillou, and B. Charreau. 2004. RhoA activation mediates phosphatidylinositol 3-kinase-dependent proliferation of human vascular endothelial cells: an alloimmune mechanism of chronic allograft nephropathy. J Am Soc Nephrol 15:2429-2439.
35. Klebanoff, C. A., L. Gattinoni, and N. P. Restifo. 2006. CD8+ T-cell memory in tumor immunology and immunotherapy. Immunol Rev 211:214-224.
36. Cerboni, C., M. Mousavi-Jazi, H. Wakiguchi, E. Carbone, K. Karre, and K. Soderstrom. 2001. Synergistic effect of IFN-gamma and human cytomegalovirus protein UL40 in the HLA-E-dependent protection from NK cell-mediated cytotoxicity. Eur J Immunol 31:2926-2935.
37. Tomasec, P., V. M. Braud, C. Rickards, M. B. Powell, B. P. McSharry, S. Gadola, V. Cerundolo, L. K. Borysiewicz, A. J. McMichael, and G. W. Wilkinson. 2000. Surface expression of HLA-E, an inhibitor of natural killer cells, enhanced by human cytomegalovirus gpUL40. Science 287:1031.
38. David, G., M. Morvan, K. Gagne, N. Kerdudou, C. Willem, A. Devys, M. Bonneville, G. Follea, J. D. Bignon, and C. Retiere. 2009. Discrimination between the main activating and inhibitory killer cell immunoglobulin-like receptor positive natural killer cell subsets using newly characterized monoclonal antibodies. Immunology 128:172-184.
39. Moesta, A. K., P. J. Norman, M. Yawata, N. Yawata, M. Gleimer, and P. Parham. 2008. Synergistic polymorphism at two positions distal to the ligand-binding site makes KIR2DL2 a stronger receptor for HLA-C than KIR2DL3. J Immunol 180:3969-3979.
40. Pende, D., S. Marcenaro, M. Falco, S. Martini, M. E. Bernardo, D. Montagna, E. Romeo, C. Cognet, M. Martinetti, R. Maccario, M. C. Mingari, E. Vivier, L. Moretta, F. Locatelli, and A. Moretta. 2009. Anti-leukemia activity of alloreactive NK cells in KIR ligand-mismatched haploidentical HSCT for pediatric patients: evaluation of the functional role of activating KIR and redefinition of inhibitory KIR specificity. Blood 113:3119-3129.
41. Schonberg, K., M. Sribar, J. Enczmann, J. C. Fischer, and M. Uhrberg. 2011. Analyses of HLA-C-specific KIR repertoires in donors with group A and B haplotypes suggest a ligand-instructed model of NK cell receptor acquisition. Blood 117:98-107.
42. Rodgers, J. R., and R. G. Cook. 2005. MHC class 1b molecules bridge innate and acquired immunity. Nat Rev Immunol 5:459-471.
43. Sullivan, L. C., C. S. Clements, J. Rossjohn, and A. G. Brooks. 2008. The major histocompatibility complex class 1b molecule HLA-E at the interface between innate and adaptive immunity. Tissue Antigens 72:415-424.
44. Reddehase, M. J. 2002. Antigens and immunoevasins: opponents in cytomegalovirus immune surveillance. Nat Rev Immunol 2:831-844.
45. Wilkinson, G. W., P. Tomasec, R. J. Stanton, M. Armstrong, V. Prod'homme, R. Aicheler, B. P. McSharry, C. R. Rickards, D. Cochrane, S. Llewellyn-Lacey, E. C. Wang, C. A. Griffin, and A. J. Davison. 2008. Modulation of natural killer cells by human cytomegalovirus. J Clin Virol 41:206-212.
46. Mazzarino, P., G. Pietra, P. Vacca, M. Falco, D. Colau, P. Coulie, L. Moretta, and M. C. Mingari. 2005. Identification of effector-memory CMV-specific T lymphocytes that kill CMV-infected target cells in an HLA-E-restricted fashion. Eur J Immunol 35:3240-3247.
47. Moretta, L., C. Romagnani, G. Pietra, A. Moretta, and M. C. Mingari. 2003. NK-CTLs, a novel HLA-E-restricted T-cell subset. Trends Immunol 24:136-143.
48. Jabri, B., J. M. Selby, H. Negulescu, L. Lee, A. I. Roberts, A. Beavis, M. Lopez-Botet, E. C. Ebert, and R. J. Winchester. 2002. TCR specificity dictates CD94/NKG2A expression by human CTL. Immunity 17:487-499.
49. Takemoto, S., F. K. Port, F. H. Claas, and R. J. Duquesnoy. 2004. HLA matching for kidney transplantation. Hum Immunol 65:1489-1505.
50. van Bergen, J., A. Thompson, G. W. Haasnoot, J. I. Roodnat, J. W. de Fijter, F. H. Claas, F. Koning, and Doxiadis, II. KIR-ligand mismatches are associated with reduced long-term graft survival in HLA-compatible kidney transplantation. Am J Transplant 11:1959-1964.

1. A method for determining whether a cytomegalovirus infection in a transplanted patient is susceptible to induce allograft rejection comprising detecting the presence of at least one HLA-E-restricted CD8 αβ T cell population displaying reactivity against peptides derived from the leader sequences of both HCMV-UL40 protein and allogeneic classical HLA-I molecules in a blood sample of the patient, wherein the presence of said populations indicates that the cytomegalovirus infection in the transplant patient is susceptible to induce allograft rejection.

2. The method of claim 1 which further comprises the step of HLA class I typing of the transplant donor.

3. The method according to claim 2 wherein the i) the presence of at least one HLA-E-restricted CD8 αβ T cell population displaying reactivity against VMAPRTLLL (SEQ ID NO:1) in the blood sample of the patient ii) the presence of at least one allele selected from the group consisting of HLA-A*01,-A*03, -A*11, -A*29, -A*30, -A*31, -A*32, -A*33, -A*36 -A*74, -Cw*2 and -Cw*15 in the HLA typing of the transplant donor and iii) the absence of the of a HLA-C ligand for KIR2DL2 in the HLA typing of the transplant donor indicate that the cytomegalovirus infection in the transplant patient is highly susceptible to induce allograft rejection.

4. The method according to claim 2 wherein the i) the presence of at least one HLA-E-restricted CD8 αβ T cell population displaying reactivity against VMAPRTLVL (SEQ ID NO:2) in the blood sample of the patient ii) the presence of at least one allele selected from the group consisting of HLA-A*02, -A*23, -A*24, -A*25, -A*26, -A*3402, -A*43, -A*66 and -A*69 in the HLA typing of the transplant donor and iii) the absence of the of a HLA-C ligand for KIR2DL2 in the HLA typing of the transplant donor indicate that the cytomegalovirus infection in the transplant patient is highly susceptible to induce allograft rejection.

5. The method according to claim 2 wherein the i) the presence of at least one HLA-E-restricted CD8 αβ T cell population displaying reactivity against VMAPRTLIL (SEQ ID NO:3) in the blood sample of the patient ii) the presence of at least one allele selected from the group consisting of HLA-Cw*01, -Cw*03, -Cw*0401, -Cw*05, -Cw*06, -Cw*0801-03, -Cw*12, -Cw*14, -Cw*16 and -Cw*1702 in the HLA typing of the transplant donor and iii) the absence of the of a HLA-C ligand for KIR2DL2 in the HLA typing of the transplant donor indicate that the cytomegalovirus infection in the transplant patient is highly susceptible to induce allograft rejection.
